(19) United States
(12) Patent Application Publication
(10) Pub. No.: US 2006/0099176 A1
(43) Pub. Date: May 11, 2006

(54) Medicines including interferon and administering system thereof

(76) Inventor: Shinya Nakajima, Osaka (JP)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 11/320,052

(22) Filed: Dec. 28, 2005

Related U.S. Application Data
(62) Division of application No. 10/685,757, filed on Oct. 15, 2003.

(30) Foreign Application Priority Data
Oct. 25, 2002 (JP) 2002-311571

(51) Int. Cl. A61K 38/21

(52) U.S. Cl. 424/85.6; 424/85.7

(57) ABSTRACT

The present invention discloses medicines including interferon and administering systems thereof, which have characteristics of the preparation according to the number of interferon on the surface of the cell and/or 2-5AS activity induced in the cell. The administering system of the present invention includes the first step of detecting the number of interferon receptors which are prepared so that the interferon dosage is determined according to the extent of interferon receptors on the surface of the cell.

### CROSS REFERENCE TO RELATED APPLICATIONS

This application is a divisional of U.S. Ser. No. 10/685,757, filed on Oct. 15, 2003 and entitled “Medicines Including Interferon and Administering System Thereof,” the entirety of which is incorporated by reference herein.

### BACKGROUND OF THE INVENTION

1. Field of the Invention

The present invention relates generally to the medicines including interferon, and administering systems thereof. In detail, the present invention relates to the medicines including interferon, and administering systems thereof for treating chronic liver disease due to infection with hepatitis C virus (HCV), wherein the patient does not suffer from any side effects and his immune system is bolstered.

2. Description of the Related Art

Interferon (IFN) is a kind of the glycoprotein, which can be categorized into (IFN-α, and IFN-β, and IFN-r), IFN puts the cell infected with the virus into the antiviral state, wherein the proliferation of the virus is inhibited. This is called antiviral effect. IFN has more functions including the cytostatic function, the antineoplasmic function, the NK cell activation, the immunological enhancement function, and the virus exclusion actions. This is the reason why interferon is authorized to the treatments such as leukemia, malignant myeloma, hepatitis B and C, AIDS which relates to Kaposi's sarcoma or genital tumor, and renal cancer. Especially, IFN is the only basic medicine for treating chronic liver disease due to infection with HCV, and used in various manner. Currently IFN-α and IFN-β are used for the treatment of chronic hepatitis C.IFN-α is administered to the patient by muscular injection or hypodermic injection, and IFN-β is administered to the patient by intravenous injection.

Chronic hepatitis C accounts for approximately 60% of the chronic hepatitis, and has the high possibility to progress to cirrhosis and to hepatocellular carcinoma. Hepatitis C virus, the cause of this chronic hepatitis C, is an RNA virus and its gene mutates frequent like the influenza virus. Consequently, the surface antigen protein of the virus changes one after another. This means that the neutralizing antibody is not generated easily even if the immunity reaction takes place in inside the body, and that the liver cell is persistence-infected easily. Therefore, it is HCV be suppressed at the early stage, the inflammation be soothed, and the progress to cirrhosis and hepatocellular carcinoma be hindered.

The dosage dependency is admitted in the efficacy of IFN therapy, and it is said that the antiviral effect increase as the dose of IFN is higher. Then, it is standard direction for IFN-α applied to treating chronic hepatitis C to administer 3 million to 10 million IU (generally 6 million IU) daily at the initial stage followed by such treatment of three times a week. For IFN-β, 3 million to 6 million IU dosage is general (For instance, refer to patent document 1).

The virus disappearance rate of the standard directions is about 10-20%. This means that virus has disappeared in about 10-20% of patients whom the standard directions are applied to. It is a known that such as lengthening the daily administering period, and increasing dosage to the maximum does are attempted to result with no improvement on therapeutic effect, at the cost of patients' suffering from many toxic side effects, such as fever, malaise, and depression. If these side effects are strong, IFN therapy shall necessary be abandoned.

Recently, the oral medicine with an antiviral effect named “ribavirin” has been developed, and combination therapy of interferon and ribavirin is attempted. This combination treatment with interferon and ribavirin can attain to increase the virus disappearance rate up to about 30%, concluding that such combination therapy is the most reliable treatment for hepatitis C. However, toxic side effects associated with this combination treatment are very strong, and abandonment rate of this combination therapy is higher than that of IFN monotherapy. Moreover, even if IFN monotherapy or combination therapy is applied to patients with chronic hepatitis C, 70% patients have problems that HCV remains and hepatopathy continues. In addition, further difficulties with IFN therapy are that patients whose chronic liver disease progresses to cirrhosis and there are many patients who cannot endure the toxic side effects or in whom therapeutic effects are not exerted.

It is a current state to suppress hepatitis by using the medicine named Stronger Neo-Minophagen C (SNMC) for patients who did not get efficacy of such IFN monotherapy or combination therapy of interferon and ribavirin, do not have the adjustment in respect of toxic side effects, and cannot take interferon and/or ribavirin for fear of toxic side effects. When Stronger Neo-Minophagen C is used, does of 40-60 ml is administered intravenously once a day. It is known that Stronger Neo-Minophagen C contains the element of glycylryicin which has the anti-inflammatory function and improves the value indicating the extent of liver damage. While Stronger Neo-Minophagen C is cheaper than IFN and its side effects are fewer, we cannot expect that it excludes the virus completely because it does not attack the virus directly like IFN, and the patients have inconvenience that it is necessary to keep going regularly to hospital for intravenous injection.

As mentioned above, IFN is very useful compared with other medicines because IFN possesses the antiviral effect, the carcinogenesis preventive function, and the immunological enhancement function. However, it is the fact that IFN contain the problem with strong toxic side effects. The reason why side effects of IFN cannot be removed is to clarify neither detailed mechanisms in various effects such as the antiviral effect of IFN nor manifestation mechanism of side effects. Thus, the more effective therapy with slight side effect has been researched.

The behavior of IFN within the body is explained here while referring to Figure. Interferon (IFN) (1) administered to the body binds to the IFN receptor (3) on the surface of the cell. The IFN (1)-IFN receptor (3) complex (5) diffuses into the cell (7) to head for the nucleus (9). This IFN (1)-IFN receptor (3) complex (5) results in the production of 2-5AS (2′,5′-oligoadenylate synthetase) in the cell (7). As the concentration of IFN increases the number of IFN receptors decrease on the surface of the cell and 2-5AS increases.

FIG. 2 shows how IFN-α and 2-5AS are influenced by the concentration of IFN-α (Refer to Chika Kawamura et al “Two-dimensional Analysis of Production of IL-6 and TNF-α can Predict the Efficacy of IFN-α Therapy”: Hepato-Gastroenterology 1999. p. 2943). Note that this Kawamura's data is the experimental result carried out in vitro. The number on the abscissa indicate the concentration of IFN-α in the culture medium (IU/ml). The percentage on the left-ordinate indicate the decrease of IFN-α receptor as the concentration of the added IFN-α was increased, wherein the number of IFN-α receptors is defined as 100% when no IFN-α was added to the culture medium (bar chart). The result of determination of 2-5AS activity of peripheral blood mononuclear cells are expressed on the right-ordinate as one-fold when no IFN-α was added to the culture medicine (line chart).

It is apparent in FIG. 2 that the number of IFN receptors decreases and 2-5AS activity increase when the concentration of IFN-α increases. If IFN exerts the biological effects, 2-5AS is induced within the body. It is known that the higher 2-5AS activity is, the higher the antiviral effect is. As shown in FIG. 2, even if the concentration of IFN is as low as 1 IU/ml in the culture medium, 2-5AS activity increases to 2-3 fold, thus the biological effect exerted by IFN is recognized at the IFN concentration. This concentration corresponds to the amount of 5000 IU/body when the amount of blood of one person is estimated roughly as 5000 ml. That is, this fundamental experiment in vitro had the biological effect is exerted when the concentration of IFN was as low as only 5000 IU. However, it is assumed that the best concentration of IFN-α is 10000 IU/ml (50 million IU/body) according to Kawamura. Please refer Japanese Disclosure Gazett 2000-7578 for related description to this matter.

As mentioned above, when IFN is currently used for treatment of chronic liver disease due to infection with HCV, the standard dosage of IFN-α is 3 million to 10 million IU (generally 6 million IU),and that of IFN-β is 3 million to 6 million IU. There is no conception of administering IFN less than this standard dosage to the patient. It is expected to establish the effective method for treating chronic liver disease due infection with HCV, while the appearance of the toxic side effects being suppressed by taking advantage of outstanding characteristics of IFN.

The purpose of the present invention is to provide the medicines including interferon(IFN),and administering systems thereof, wherein patient does not suffer from any side effects while his immune being bolstered. Especially, the purpose of the present invention is to provide systems for treating chronic liver disease due to infection with HCV, which comprises the preparation of the medicines including IFN with no toxic side, and administration of these medicines to the patient at appropriate intervals.

### SUMMARY OF THE INVENTION

Taking into account the principle shown in FIG. 1 and the fundamental data shown in FIG. 2, the inventor of the present invention considered that the IFN dosage below well-known level exerts its efficacy satisfactorily, and he applied the small amount IFN therapy to patients who consented to this therapy.

As result, the inventor has discovered that above-mentioned purposes have been achieved by providing the medicines including IFN, which have characteristics of the preparation according to the number of IFN receptors on the surface of the cell and/or 2-5AS activity induced in the cell. Preferably, the medicines of the present invention are prepared so that the IFN dosage is less than 3 million IU in each administration or the IFN dosage is the amount where expected outstanding effects of IFN are exerted and it does not exceed 3 million IU. Any kinds of IFN are suitable for the present invention, but the use of IFN-α or IFN-β is preferred. For instance, the medicines of the present invention are used for treatment of chronic liver disease due to infection with HCV.

Within further embodiments, the present invention provides administering systems which have characteristics of the preparation of the medicines including IFN according to the number of IFN receptors on the surface of the cell and/or 2-5AS activity induced in the cell, and the administration of them. Preferably, the medicines of the present invention are prepared so that the IFN dosage is less than 3 million IU in each administration or the IFN dosage is the amount where expected outstanding effects of IFN are exerted and it does not exceed 3 million IU. Any kinds of IFN are suitable for the present invention, but the use of IFN-α or IFN-β is preferred. For instance, administering systems of the present invention are used for treatment of chronic liver disease due to infection with HCV. A desirable administering frequency is three times a week.

The biological effect exerted by IFN is closely related to the number of IFN receptors and the value of 2-5AS activity. The inventor has reached the recognition that the IFN dosage is adequate to bind to IFN receptors, and the existence of IFN more than the necessity cause toxic side effects. Based on this idea, the inventor considered that the IFN therapy for chronic liver disease due to infection with HCV might become possible with no toxic side effect through the convectional IFN administering system was devised.

A large does of IFN in order to exclude the cause virus completely would impose the heavy burden concerned with physical, time and money on the patient, such as side effects, expensive doctor's fee, and frequent go to hospital regularly. The inventor judged that it is the best therapy so that toxic side effects did not exert at the time immunological enhancement function was induced within the patient's body. It is clear that the present invention can be applied for treatment of other diseases widely because the feature of the present invention is to predict the optimum IFN dosage quantitatively.

Briefly, the present invention provides methods for preparation of the medicines including IFN, comprising the first step of detecting the number of IFN receptors on the surface of the cell, and the second step of determining the IFN dosage according to the extent of IFN receptors on the surface of the cell. The first detecting step includes measuring the value that corresponds to the amount of 2-5AS induced in the cell. In addition, it is preferred to prepare the medicines of the present invention so that the IFN dosage is less than 3 million IU in each administration. It is preferred that the minimum IFN dosage is more than the amount where expected outstanding effects of IFN are exerted.

Moreover, the present invention provides administering systems of the medicines including IFN, comprising the first step of detecting the number of IFN receptors on the surface of the cell, and the second step of administering the medicines including IFN which are prepared so that the IFN dosage is determined according to the extent of IFN receptors on the surface of the cell. The detecting step includes measuring the value that corresponds to the amount of 2-5AS induced in the cell. The administering interval of the medicines of the present invention is preferably twice or more a week. IFN used for the present invention includes IFN-α or IFN-β. The present invention can be typically used to treat chronic liver disease due to infection with HCV.

### BRIEF DESCRIPTION OF THE DRAWINGS

For the purpose of illustrating the invention, the drawings show a form of the invention that is presently preferred. However it should be understood that the present invention is not limited to the precise arrangements and instrumentalities shown in the drawings, wherein:

FIG. 1 is a schematic illustration which outlines the behavior of IFN within the body.

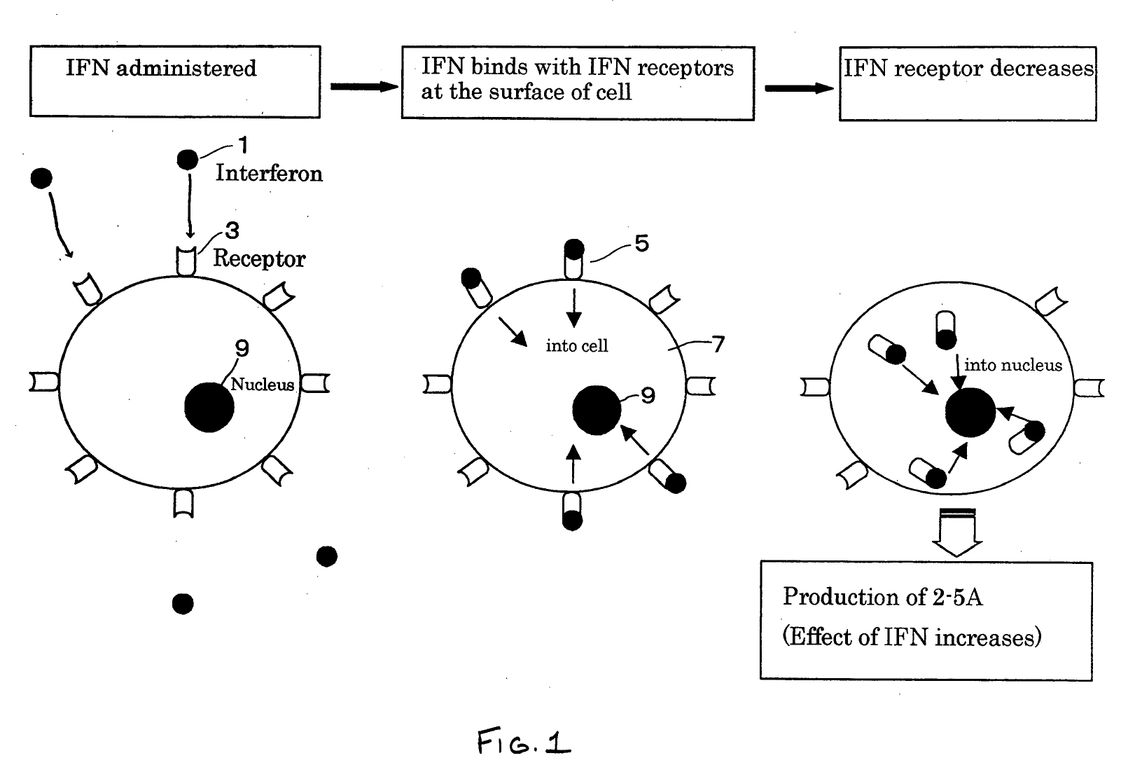

FIG. 2 is a graph showing how IFN-α and 2-5AS are influenced by the concentration of IFN-α.

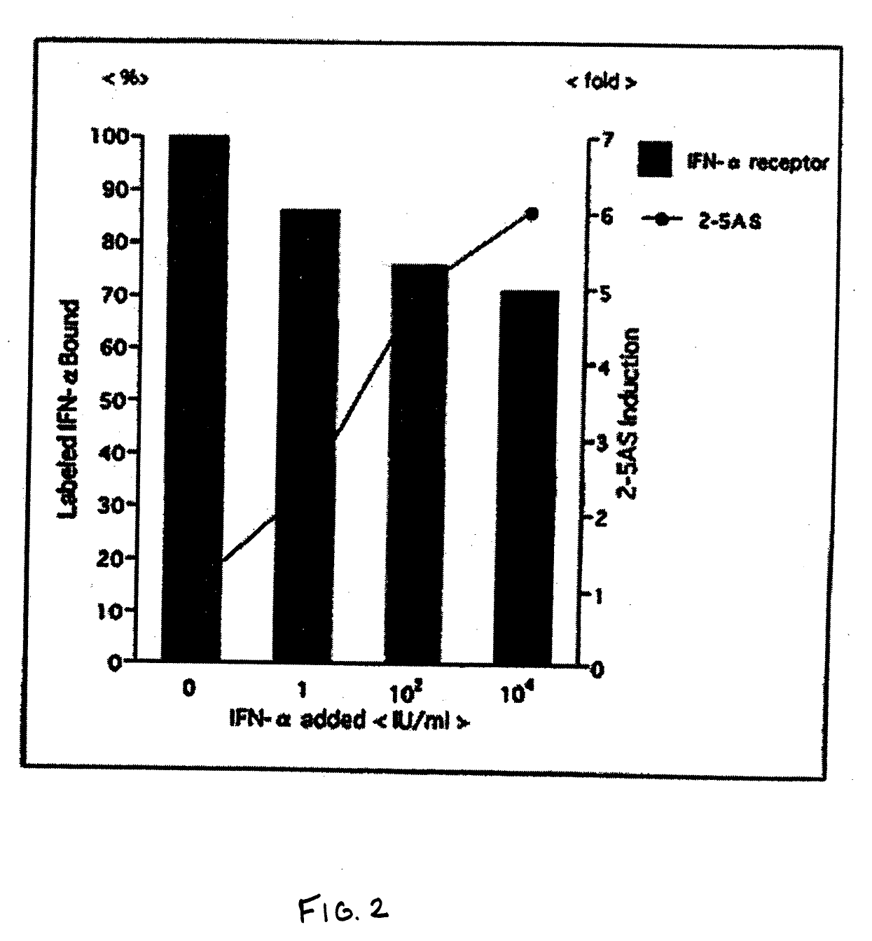

FIG. 3 is a graph showing the efficacy of the medicines including IFN of various dosage of the present invention.

FIG. 4 is a graph showing the efficacy of the medicine including IFN of 100 thousand IU of the present invention.

### DETAILED DESCRIPTION OF THE INVENTION

Within on aspect of the present invention, the medicines including IFN prepared so that the IFN dosage is less than 3 million IU are administered to the patient by injection at appropriate intervals. Any kinds of IFN can be used, but IFN-α is preferred. Notes that IFN-β exerts the same efficacy as does IFN-α undoubtedly because IFN receptor IFN-α and that IFN-β are common. The medicines including IFN are administered by the muscular injection, the hypodermic injection, or the intravenous injection(including the intravenous drip).

The IFN dosage is less than 3 million IU in each administration, and preferably, the IFN dosage is the amount where expected outstanding effects of IFN are exerted and it is less than 1 million IU. The IFN dosage should be determined properly by considering the kinds of IFN used, the administering interval, patient's condition, and so on. When IFN-α is used, the IFN dosage is preferably less than 300 thousand IU, and 300 thousand to 100 thousand IU is preferred most. The minimum is determined so that the efficacy of the therapy is exerted in the patient, however it is generally over 10 thousand IU.

Now, the administering interval is considered. It has already been understood that the number of IFN receptors on the surface of the cell decreases to about 50% by the first IFN administration in vivo, and this decreased level remains approximately constant while IFN is kept on administering daily. After IFN therapy is discontinued, the time taken to reach the original number of IFN receptors is from two to three days. That is to say, even if IFN is administered daily, all of the IFN does not contribute to exertion of the efficacy because the number of IFN receptors decreases to 50%. Thus, it is more effective that the following administration is done when the number of IFN receptors reverts to the original level on the surface of the cell, two or three days after the preceding administration. Oppositely, it has already been found that the biological effect of IFN is not exerted by the administration of present medicines once a week, no matter how dosage is increased. Therefore, the administering interval of the present invention is preferred to be every two or three days, or to be three times a week.

In the present invention, the number of IFN receptors on the surface of the cell can be detected by measuring the value that is equivalent to the amount of 2-5AS induced in the cell. Other appropriate and well-known methods can be used. As mentioned above, it is also possible to detect the number of IFN receptors on the surface of the cell by measuring any other marker as well as 2-5AS if the correlation between the number of IFN receptors and the marker is obtained.

Since an excessive IFN dosage not accepted to the IFN receptor cause toxic effects and the high pharmaceutical expense, IFN is administered while the number of IFN receptors on the surface of the cell is detected in the present invention. Experiments have been performed based on this idea, and consequently, the inventor of the present invention found that the IFN dosage below 3 million IU in each administration is adequate for the purpose of the invention. The IFN dosage below 1 million IU in each administration is preferred. When IFN-α is used, the IFN dosage is preferably less than 300 thousand IU. But the IFN dosage should be determined properly by considering the kinds of IFN used, the administering interval, patient's condition, and so on.

The embodiment of the present invention is explained in detail as follows with figures containing various data.

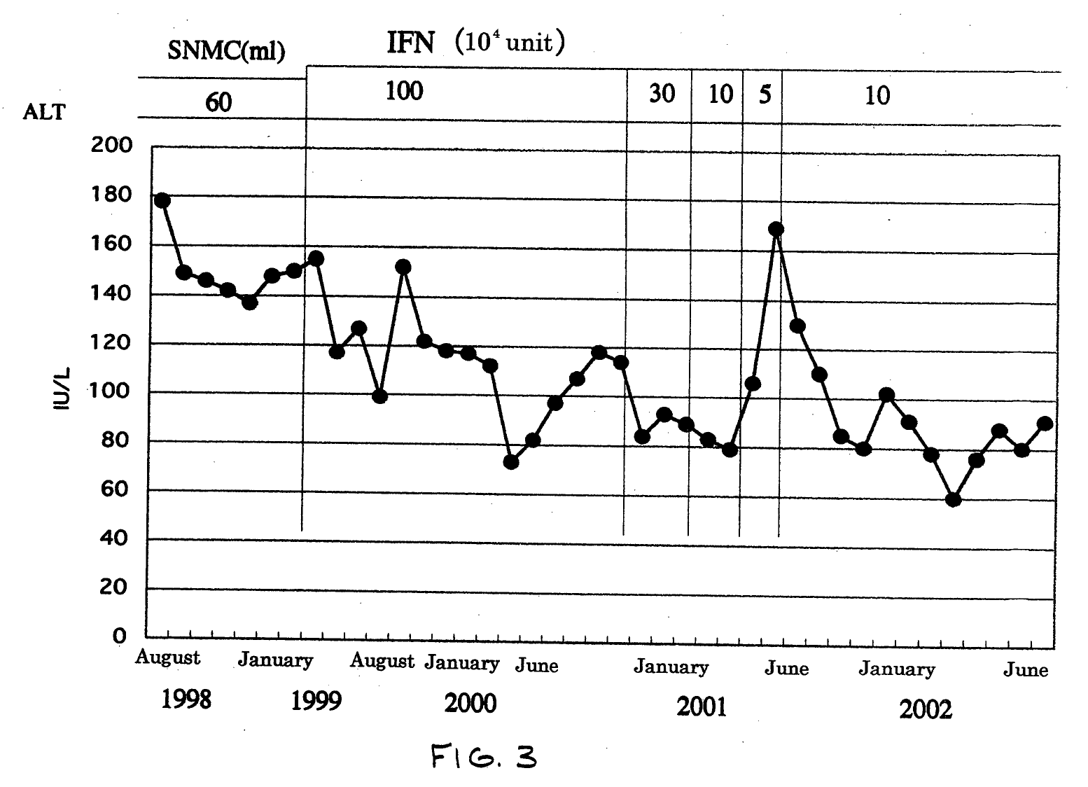

FIG. 3 shows the result of administering the medicines including IFN-α instead of SNMC (strong neo minofagen C) to the patient who had previously been administered 60 ml SNMC, where the medicines were prepared so that the IFN dosage was less than 1 million IU. ALT value is plotted as the GPT (glutamic-pyruvic transaminase) value generally measured in hemodiagnosis of physical checkup, and indicates increased quantification of this enzyme in serum caused by leakage of this enzyme out of damaged liver cells or hepatocellular necrosis. A normal value is 40 IU/L or less, and it is assumed a typical index of hepatocellular damage. In this experiment of the present invention, the medicines including IFN prepared so that the IFN-α dosage may become 1 million IU, 300 thousand IU, 100 thousand IU, and 50 thousand IU were used.

The medicines including IFN of the present invention are prepared by the original method that comprises of the dilution of a commercially available IFN ampoule with the physiological saline and the feeze-storage of these diluted medicines, and then a necessary amount of these medicines are used when necessary. For example, the medicines including IFN of 100 thousand IU is prepared by mixing and stirring the physiological saline 14 ml and 1 ml of 3 million IU Sumiferon interferon (liquid) available from Sumitomo Pharmaceutical Corporation, Japan at the room temperature in the sterilized injection cylinder for 20 ml to make 15 times diluted solution. An amount of 0.5 ml of this diluted solution is poured into a sterilized injection cylinder for 1 ml in order to make 30 cylinders contained IFN of 100 thousand IU respectively. The sterilized injection needle of 24 G is applied to each of these injection cylinders, and then these injection cylinders are preserved immediately in the frozen warehouse of −40□. The medicine including IFN of 300 thousand IU Sumiferon interferon (liquid) available from Sumitomo Pharmaceutical Co. at the room temperature in the sterilized injection cylinder for 20 ml to make 10 times diluted solution of 6 million IU Sumiferon. An amount of 0.5 ml of this diluted solution is poured into a sterilized injection cylinder for 1 ml in order to make 20 cylinder contained IFN of 300 thousand IU respectively.

The sterilized injection needle of 24 G is applied to each of these injection cylinders, and then these injection cylinders are preserved immediately in the frozen warehouse of −40□ by the above method. After one week or two weeks freezing, no decrease of interferon IU of the present medicines was observed by measuring the measurement of antiviral activity by a 50% cytopathic effect (CPE) inhibition and dye uptake method. For usage of the medicine including IFN, the freeze-stored injection cylinder should be dissolved at the room temperature. The medicines including IFN of the present invention were administered to the patient three times a week by the intra-muscular injection. Even the powdery interferon (for instance, Intron interferon available from Schering Corporation, Keniworth, N.J.) can be used to prepare the medicines including IFN of the present invention according to the procedure previously described, where the interferon is diluted to the appropriate concentration by mixing and stirring with the physiological saline.

In FIG. 3, the ALT value decreased gradually after the administration of the medicine including IFN of 1 million IU had been started (In January, 1999), and it was kept low even if the medicine including IFN of 300 thousand IU (In October, 2000) and the medicine including IFN of 100 thousand IU (In February, 2001) had been administered. This shows that the administration of the medicine including IFN of less than 1 million IU, even 100 thousand IU, to the patient has definitely exerted the biological effect of interferon in vivo. Especially, it has been understood that the medicines including IFN of 100 thousand IU to 300 thousand IU are preferred, and the medicine including IFN of 100 thousand IU is the most preferable if the pharmaceutical expense is taken into consideration.

The increase of the ALT value is seen in this graph by the change the medicine including IFN of 100 thousand IU to that of 50 thousand IU. However, the minimum amount of the IFN dosage depends on the patient's symptom level, condition, and administering purpose. Therefore, the medicine including IFN of 50 thousand IU should not be defined as the lower limit of the present invention because this is only one example. It is necessary to prepare the medicine including IFN where the lower limit of IFN dosage may be defined from the purpose of present invention so that the efficacy of dosage is certainly exerted or patient's condition does not deteriorate.

Similarly the IFN dosage of 1 million IU to 3 million IU can be properly determined by considering patient's conditions if necessary without departing from the scope of the present invention.

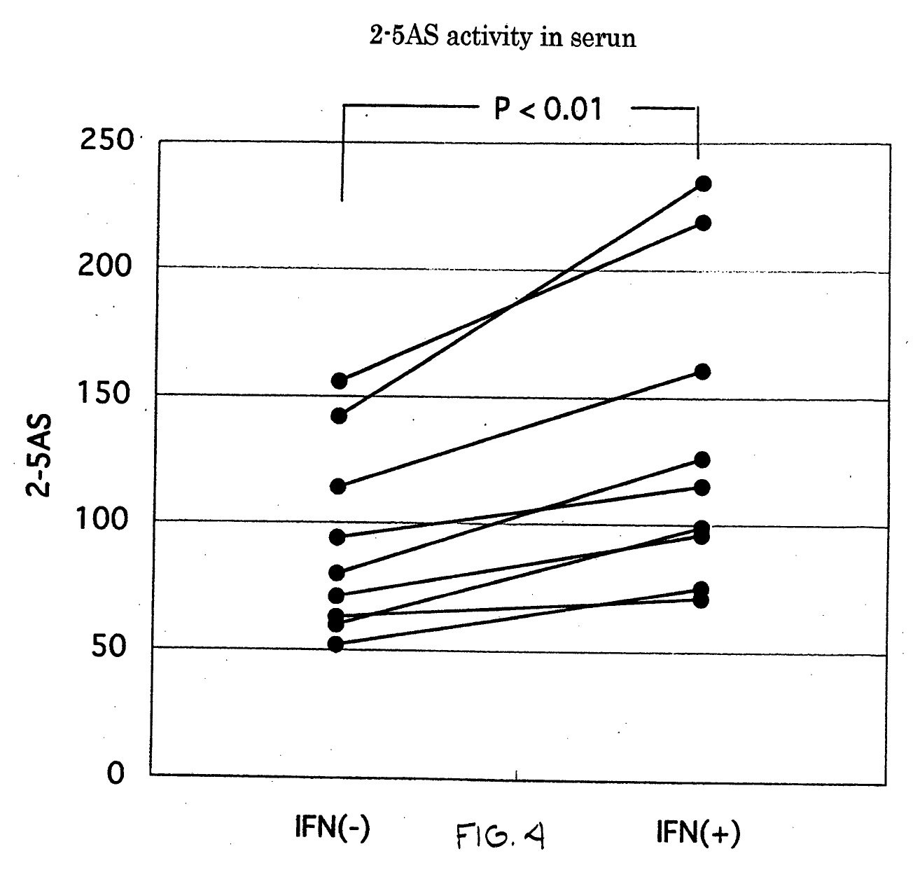

FIG. 4 shows the measurement result of 2-5AS activity when the medicines including IFN-α of 100 thousand IU were administered to nine patients three times a week. On abscissa axis, IFN (−) means the results before interferon (IFN) administration and IFN (□) means those after IFN administration. The numbers on the ordinate 2-5AS activities (pmol/dl). Most of the administered patient suffered from terminal cirrhosis or progressed hepatitis. Moreover, though many of these patients had tried various therapies, these therapies had not been effective for them and their liver diseases had been progressing. In brief, they were patients in whom therapeutic effect were not exerted easily, although the patients included the person who had no prior treatment with any medicines before administration of the medicines including IFN of the present invention. Administration began after explaining the content of therapy enough and obtaining the consent of the patients.

As shown in FIG. 4, it was observed that 2-5AS activities after administration increased in all the patients compared with those of before administration of the medicine including IFN of the present invention. Therefore, administration of the medicine including IFN of 100 thousand IU caused the biological effect, and then, the therapeutic efficacy had been clarified.

The administration interval of the therapy in FIG. 4 was three times a week, but the present invention does not limited to this interval. The preferable administration intervals are from once a week to four times a week, and shorter intervals are within the scope of the present invention if the therapeutic efficacy is recognized concerning with patient's condition and IFN dosage.

The present invention removes the side effect from patient and contributes to eliminate the physical burden on patient remarkably. The invention has the pronounced effects to suppress the toxic side effect, that has been the problem in interferon therapy, and to induce the immunological enhancement function characteristic of interferon inside the patient's body.

The present invention is very useful because of having the effects characteristic of interferon and offering patient cheap treatment. It has been pointed out that IFN therapy is expensive. On the other hand, Stronger Neo-Minophagen C is cheap, but IFN is more excellent than this medicine in respect of the function because IFN has the virus suppressible function and the immunological enhancement function. The medicines including the small amount of IFN of the present invention is not expensive compared with Stronger Neo-Minophagen C therapy. It depends on what kind of interferon is used. The invention not only offers effective treatment but also contributes to eliminate the money burden on patient remarkably by the treatment expense reduction.

The present invention can not only offer the hindrance of chronic hepatitis C and cirrhosis progress by the long-term administration but also gives the possibility of the prevention of hepatocellular carcinoma by the carcinogenesis suppressible function. The present invention provides the inexpensive and side-effect-free therapy, thus enable the long-term administration.

It can be said that the medical contribution of the present invention is surprising since the invention provides new medicines including IFN and physical burden on the patient remarkably. When the current state used in the treatment field of interferon is considered, the contribution is inscrutable.

### DESCRIPTION OF THE SIGNS

1 interferon (IFN)

3 interferon receptor (IFN receptor)

5 complex

7 cell

9 nucleus

1. An administering system of medicines including interferon, comprising a first step for detecting the number of interferon receptors on a surface of the cell, and a second step for administering the medicine including interferon being prepared so that an interferon dosage is determined according to an extent of IFN receptors on the surface of the cell.

2. The administering system of the medicines including interferon according to claim 1 wherein said first step is characterized by measuring the value that corresponds to the amount of 2-5AS induced in the cell.

3. The administering system of the medicines including interferon according to claim 1 wherein said interferon dosage is less than 3 million IU in each administration.

4. The administration system of the medicines including interferon according to claim 1 wherein said medicines including interferon is administered twice or more a week.

5. The administering system of the medicines including interferon according to claim 4 wherein said medicines including interferon is not administered daily.

6. The administering system of the medicines including interferon according to claim 1 wherein said interferon includes IFN-α or IFN-β.

7. The administering system of the medicines including interferon according to claim 6 wherein said medicines including interferon is used to treat chronic liver disease due to infection with HCV.
